(19) United States
(12) Patent Application Publication
(10) Pub. No.: US 2024/0166576 A1
(43) Pub. Date: May 23, 2024

(54) ADIABATICALLY CONDUCTED PROCESS FOR THE PRODUCTION OF 1,3-BUTADIENE FROM MIXTURES OF ETHANOL AND ACETALDEHYDE

(71) Applicant: SYNTHOS DWORY 7 SPOLKA Z OGRANICZONA ODPOWIEDZIALNOSCIA SPOLKA JAWNA, OSWIECIM (PL)

(72) Inventors: Ewelina JANKOWIAK, OSWIECIM (PL); Szymon SKOWRONEK, OSWIECIM (PL); Magdalena SITKO, OSWIECIM (PL); Wojciech GURGACZ, OSWIECIM (PL)

(21) Appl. No.: 18/552,765

(22) PCT Filed: Mar. 31, 2022
(86) PCT No.: PCT/EP2022/058731

§ 371 Date: Sep. 27, 2023

(30) Foreign Application Priority Data
Apr. 1, 2021 (EP) 21461531.2

(51) Int. Cl. C07C 1/207; C07C 45/29

(52) U.S. Cl. CPC C07C 1/2072; C07C 45/29; C07C 2521/08; C07C 2523/20

(57) ABSTRACT

The invention relates to a process for the production of 1,3-butadiene comprising reacting a feed comprising ethanol and acetaldehyde in a 1,3-butadiene producing reactor having at least one adiabatic reaction zone. Moreover, the invention relates to a process for the production of 1,3-butadiene from ethanol ater comprising i. producing acetaldehyde from ethanol in an acetaldehyde producing reactor, and ii. producing 1,3-butadiene from ethanol and acetaldehyde in a 1,3-butadiene producing reactor. The invention further relates to a plant for the production of 1,3-butadiene comprising at least one 1,3-butadiene producing reactor producing 1,3-butadiene from ethanol and acetaldehyde. Finally, the invention relates to a plant for the production of 1,3-butadiene from ethanol, comprising i. an acetaldehyde producing reactor, and ii. a 1,3-butadiene producing reactor.

The invention relates to a process for the production of 1,3-butadiene comprising reacting a feed comprising ethanol and acetaldehyde in a 1,3-butadiene producing reactor having at least one adiabatic reaction zone. Moreover, the invention relates to a process for the production of 1,3-butadiene from ethanol comprising i. producing acetaldehyde from ethanol in an acetaldehyde producing reactor, and ii. producing 1,3-butadiene from ethanol and acetaldehyde in a 1,3-butadiene producing reactor. The invention further relates to a plant for the production of 1,3-butadiene comprising at least one 1,3-butadiene producing reactor producing 1,3-butadiene from ethanol and acetaldehyde. Finally, the invention relates to a plant for the production of 1,3-butadiene from ethanol, comprising i. an acetaldehyde producing reactor producing acetaldehyde from ethanol, and ii. a 1,3-butadiene producing reactor producing 1,3-butadiene from ethanol and acetaldehyde.

1,3-Butadiene is one of the key chemicals in the polymer industry and is mainly used to manufacture synthetic rubbers. In a classic approach, 1,3-butadiene is produced on industrial scale via steam cracking of naphtha, and is separated from the effluent by extractive distillation. However, major disadvantages of this process are a high energy consumption and the reliance on fossil fuel feedstock. The risk of fossil fuel depletion as well as the increasing requirements for environmental protection drive the search for lower energy-consuming and more environmentally-benign routes for olefins production, preferably based on renewable resources, such as biomass.

Sustainable 1,3-butadiene may be produced from butanediol obtained by fermentation (WO 2009/151342 A1, WO 2017/198503 A1).

U.S. Pat. No. 9,884,800 B2, also published as US 2017/0342009 A1, discloses the coproduction of 1,3-butadiene and methyl ethyl ketone from preheated 2,3-butanediol. It does not disclose any catalysts that are directly suitable for the production of 1,3-butadiene from ethanol or from ethanol-acetaldehyde mixtures.

Also, Global Bioenergies as well as Genomatica and Braskem discovered that microorganisms are prone to converting sugar to 1,3-butadiene (U.S. Pat. No. 9,169,496 B2, US 20160369306 A1, AU 2012212118 B2). However, 1,3-butadiene productivity in those processes is too low to gain importance on an industrial scale.

CN 103772117 B teaches the production of 1,3-butadiene by oxidative (exothermic) dehydrogenation of butene.

Economic and environmental considerations have led to ethanol as one of the most promising sustainable feedstocks for 1,3-butadiene production. Two routes for the chemical conversion of ethanol to 1,3-butadiene conversion exist: the so-called one step (Lebedev) process and the two step (Ostromislensky) process. The one step process includes the direct catalytic conversion of gaseous ethanol to 1,3-butadiene. The two step process divides the reaction into two stages—i) partial dehydrogenation of ethanol to acetaldehyde and ii) conversion of a mixture of ethanol and acetaldehyde to 1,3-butadiene.

The conversion of a mixture of ethanol and acetaldehyde to 1,3-butadiene is an endothermic reaction. Maintaining a temperature in the reactor that delivers sufficient energy for the optimal conversion of the substrates to 1,3-butadiene is essential. Thus, carrying out the conversion of a mixture of ethanol and acetaldehyde to 1,3-butadiene via isothermal processes over dedicated catalysts is well known in the literature and many modifications of isothermal processes have been reported. When the conversion of a mixture of ethanol and acetaldehyde to 1,3-butadiene is carried out under isothermal conditions, the reactor and catalyst therein are heated by means of a heat transfer medium, to maintain a relatively constant temperature that is high enough to allow for the endothermic conversion of ethanol/acetaldehyde mixtures to 1,3-butadiene to take place. However, the use of heat transfer media, so as to provide the high reaction temperatures required especially for regeneration, such as molten salt fluids, is expensive and makes the reactor set-up more complicated. Regeneration at a temperature of up to 550° C. is typically required to refresh (rejuvenate) the catalyst, under (at least in part) oxidative conditions, whereas the reaction of ethanol with acetaldehyde to 1,3-butadiene is typically performed at a temperature of 320 to 420° C., such as about 350° C. Moreover, isothermal reactors are often complicated in terms of construction as they are often multi-tubular reactors. Also, reactor maintenance is more difficult when employing the typical equipment used for isothermal processes, due to the presence of the heat transfer devices. This is particularly laborious because the life-time of typical catalysts for the production of 1,3-butadiene, such as tantalum catalysts, is relatively short, and the catalyst loading needs to be changed regularly, e.g. after about 1 to 2 years.

Hence, there is a need for providing a process for the production of 1,3-butadiene from mixtures of ethanol and acetaldehyde that is more economical and allows a simpler reactor set-up and maintenance.

### SUMMARY OF THE INVENTION

According to the present invention, it was surprisingly found that the conversion of mixtures of ethanol and acetaldehyde to 1,3-butadiene can be carried out under adiabatic conditions, which is more economical and allows a simpler reactor set-up and maintenance.

Due to the endothermic nature of the conversion of the mixture of ethanol and acetaldehyde to 1,3-butadiene, an effective, uniform, and easily-controllable supply of heat to the reaction zone is one of the key factors for proper reactor design. To meet these requirements, the reactor must be characterised by a high ratio of heat transfer area to reaction volume. Thus, a typical reactor design for such application is a multitube fixed-bed reactor of the shell-and-tube heat exchanger type, where a heating medium flows through the shell and the reactants flow through the small diameter tubes (loaded with catalyst grains). Such multitube reactor is very challenging to design, in particular when a heating medium is required that is suitable for the high temperature needed for regenerating the catalyst. Furthermore, such kind of reactor is challenging to operate and to maintain, in particular when having to replace used catalyst with new catalyst. Because the conversion of the mixture of ethanol and acetaldehyde to 1,3-butadiene is carried out according to the present invention in an adiabatic thermal mode, heat supply is considered separately from reactor design, and the reactor and catalytic zone therein can be shorter and have a larger diameter. For instance, reactors of the adiabatic tubular fixed-bed type, as used in accordance with the invention, provide a simple design, are of straightforward construction and allow easy operation and maintenance.

Thus, in a first aspect, the present invention relates to a process for the production of 1,3-butadiene comprising reacting a feed comprising ethanol and acetaldehyde in a 1,3-butadiene producing reactor having at least one adiabatic reaction zone, the adiabatic reaction zone comprising a supported catalyst and producing 1,3-butadiene.

In a second aspect, the present invention relates to a process for the production of 1,3-butadiene from ethanol, comprising
i. producing acetaldehyde from ethanol in an acetaldehyde producing reactor having a reaction zone, the reaction zone of the acetaldehyde producing reactor comprising a supported or unsupported (bulk) catalyst, and
ii. producing 1,3-butadiene according to the process as described herein (with regard to the first aspect of the invention),
preferably wherein the reaction zone of the acetaldehyde producing reactor is an isothermal reaction zone.

Moreover, in a third aspect, the invention relates to a plant for the production of 1,3-butadiene comprising at least one reactor for producing 1,3-butadiene, the reactor for producing 1,3-butadiene having
a) at least one zone for producing 1,3-butadiene, the zone comprising a supported catalyst for producing 1,3-butadiene from ethanol and acetaldehyde, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react the ethanol and the acetaldehyde under adiabatic conditions,
the reactor for producing 1,3-butadiene further having
c) means for regenerating the supported catalyst for producing 1,3-butadiene, preferably wherein the means for regenerating the supported catalyst for producing 1,3-butadiene comprise
x) means for feeding a flow comprising inert gas into the reactor for producing 1,3-butadiene, and
y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.

Finally, and in a fourth aspect, the invention relates to a plant for the production of 1,3-butadiene from ethanol, comprising
i. at least one reactor for producing acetaldehyde from ethanol, the reactor for producing acetaldehyde from ethanol having
   a) at least one zone for producing acetaldehyde from ethanol, the zone for producing acetaldehyde from ethanol comprising a supported or unsupported (bulk) catalyst for producing acetaldehyde, and
   b) means for feeding a feed comprising ethanol into the reactor for producing acetaldehyde; and
ii. at least one reactor for producing 1,3-butadiene, the reactor for producing 1,3-butadiene having
   a) at least one zone for producing 1,3-butadiene, the zone comprising a supported catalyst for producing 1,3-butadiene from ethanol and acetaldehyde, and
   b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react ethanol and acetaldehyde under adiabatic conditions,
the reactor for producing 1,3-butadiene further having
   c) means for regenerating the supported catalyst for producing 1,3-butadiene, preferably wherein the means for regenerating the supported catalyst for producing 1,3-butadiene comprise
   x) means for feeding a flow comprising inert gas into the reactor for producing 1,3-butadiene, and
   y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions,
preferably wherein the reaction zone of the acetaldehyde producing reactor is an isothermal reaction zone.

### DETAILED DESCRIPTION OF THE INVENTION

### 1) Process for the Production of 1,3-Butadiene

According to a first aspect of the invention, the process for the production of 1,3-butadiene comprises reacting a feed comprising ethanol and acetaldehyde in a 1,3-butadiene producing reactor having at least one adiabatic reaction zone, the adiabatic reaction zone comprising a supported catalyst and producing 1,3-butadiene.

According to the present invention, the heat energy required for the (endothermic) reaction of the mixture of ethanol and acetaldehyde, to give 1,3-butadiene, is supplied to the adiabatic reaction zone only by the feed supplied to the adiabatic reaction zone. Said feed supplied to the adiabatic reaction zone is consequently heated to a suitable temperature by heating means, before the contacting of the feed supplied to the adiabatic reaction zone with the supported catalyst takes place.

The heating means for increasing the temperature of the feed supplied to the adiabatic reaction zone may be, for example, a heat exchanger or a heated inert packing separating two adiabatic reaction zones within one reactor.

According to the invention, a heat exchanger train is designed specifically to supply heat to the feed that acts as heat carrier, and the reactor design focuses on a diminution of heat losses:
In reaction stage a), the feed comprising ethanol and acetaldehyde acts as heat carrier for effecting the endothermic reaction to 1,3-butadiene under adiabatic conditions. The reactant heating means are sufficient to react ethanol and acetaldehyde under adiabatic conditions, when the heated feed comprising ethanol and acetaldehyde contacts the supported catalyst for producing 1,3-butadiene.
In regeneration stage b), the respective heated gas flows act as heat carrier for regenerating the supported catalyst under adiabatic conditions. The regenerant heating means are sufficient so that the heated gas flow, when it contacts the supported catalyst, regenerates the supported catalyst under adiabatic conditions.

The feed supplied to the adiabatic reaction zone comprises a feed comprising ethanol and acetaldehyde and, optionally, additional feed comprising acetaldehyde.

The effluent from the reaction zone, or, if several (n) reaction zones are used, the effluent from the n^{th} reaction zone, is separated and ethanol and acetaldehyde are purified to a certain purity level, before recycling them. Ethanol from the effluent may be recycled to the reaction zone producing acetaldehyde, or to the (first or any subsequent) reaction zone producing 1,3-butadiene, or to both the reaction zone producing acetaldehyde and the (first or any subsequent) reaction zone producing 1,3-butadiene. Acetaldehyde from the effluent may be recycled to the (first or any subsequent) reaction zone producing 1,3-butadiene.

Since, in the process according to the invention, the heat energy required for the endothermic reaction of the mixture of ethanol and acetaldehyde, to give 1,3-butadiene, is supplied to the adiabatic reaction zone by the feed supplied to the reaction zone, no additional heat supply to the adiabatic reaction zone is required. It was surprisingly found that, even though the temperature decreases along the adiabatic reaction zone due to the endothermic effect of the reaction, the conversion of ethanol and acetaldehyde to 1,3-butadiene can be carried out efficiently without the provision of additional heating means for maintaining a constant temperature in the reaction zone, as long as the feed comprising ethanol and acetaldehyde has a suitably high temperature when entering the adiabatic reaction zone comprising a supported catalyst and producing 1,3-butadiene. The process according to the present invention is advantageous since it allows a much simpler and economical reactor set-up.

Preferably, the reaction zone or several reaction zones producing 1,3-butadiene and operating under adiabatic conditions are designed in terms of the temperature drop observed in each individual reaction zone such that each individual reaction zone operates within the temperature range providing good activity, conversion and selectivity towards 1,3-butadiene.

With the process according to the present invention as described herein, a mixture of ethanol and acetaldehyde can be converted to 1,3-butadiene with a conversion rate of about 35 to 45% and a selectivity to 1,3-butadiene of 70 to 75%.

Preferably, in the process according to the invention, the feed to the adiabatic reaction zone comprises at least 40 wt. %, more preferably at least 70 wt. %, of ethanol based on the total weight of the feed.

According to a preferred embodiment, the ethanol starting material used in the process according to the invention is aqueous ethanol, preferably is at least 80 wt. % aqueous ethanol, more preferably at least 90 wt. % aqueous ethanol, based on the total weight of the ethanol starting material.

According to another preferred embodiment, the ethanol starting material used in the process according to the invention comprises more than 90 wt. %, preferably more than 95 wt. %, more preferably more than 97 wt. %, most preferably more than 98 wt. % of ethanol, based on the total weight of the ethanol starting material.

Preferably, the feed comprises at least 12.5 wt. %, more preferably at least 20 wt. %, of acetaldehyde based on the total weight of the feed.

The acetaldehyde fed into the 1,3-butadiene producing reactor may be produced by an acetaldehyde producing reactor producing acetaldehyde from ethanol as described herein further below.

Alternatively, the acetaldehyde may be obtained from the workup of the effluent from a reaction zone or reactor producing 1,3-butadiene.

According to a preferred embodiment of the process according to the invention, the supported catalyst comprises one or more of tantalum, zirconium, niobium, hafnium, titanium, and tin, in particular tantalum.

Preferably, the supported catalyst comprises tantalum in an amount of from 0.1 to 10 wt. %, preferably from 0.5 to 5 wt. %, more preferably from 2 to 3 wt. %, calculated as Ta₂O₅ and based on the total weight of the supported catalyst.

Preferably, the supported catalyst comprises one or more of tantalum, zirconium, niobium, and hafnium.

According to a preferred embodiment, the support of the supported catalyst is selected from the group consisting of ordered and non-ordered porous silica supports, aluminium oxide supports, aluminosilicate supports, clays, other porous oxide supports, and mixtures thereof.

Preferably, the support of the supported catalyst is a silica support, more preferably an ordered or non-ordered porous silica support.

Preferably, the support of the supported catalyst has a specific surface area (SSA) in a range of from 130 to 550 m²/g, more preferably in a range of from 190 to 350 m²/g. Within the framework of the present text, the term “specific surface area” means the BET specific surface area (in m²/g) determined by the single-point BET method according to ISO 9277:2010, complemented by, if applicable, ISO 18757:2003.

Preferably, the support of the supported catalyst has an average pore diameter in a range of from 30 to 300 Å (determined by the method of Barrett, Joyner and Halenda).

Preferably, the support of the supported catalyst has a pore volume in a range of from 0.2 to 1.5 ml/g (determined by the method of Barrett, Joyner and Halenda).

More preferably, the support of the supported catalyst is a silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

Most preferably, the support of the supported catalyst is an ordered or non-ordered porous silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

According to a preferred embodiment of the process according to the invention, the molar ratio of ethanol to acetaldehyde in the feed to the adiabatic reaction zone is in the range of from 1 to 7, preferably 1.5 to 5, more preferably 2 to 4, in particular 2.5 to 3.5, such as about 3.

Preferably, the weight hourly space velocity (WHSV) in the adiabatic reaction zone is in the range of from 0.5 to 10 h⁻¹, more preferably from 1.5 to 4 h⁻¹, most preferably from 2 to 3 h⁻¹.

Most preferably, the WHSV is adjusted such that the molar ratio of ethanol to acetaldehyde in the effluent from the adiabatic reaction zone is at least 20%, preferably at least 30%, higher than the molar ratio of ethanol to acetaldehyde in the feed.

As outlined above, the heat energy required for the (endothermic) reaction of the mixture of ethanol and acetaldehyde, to give 1,3-butadiene, is supplied to the adiabatic reaction zone only by the feed supplied to the adiabatic reaction zone. The temperature drop depends on conversion and insulation of the reactor: heat losses. Typically, the progress of the endothermic conversion of the mixture of ethanol and acetaldehyde to 1,3-butadiene causes a temperature drop of about 30 to 100° C. along the length of the adiabatic reaction zone depending on conversion and reaction conditions. In order to maintain high efficiency, the feed must be preheated and act as a heat carrier to supply the necessary energy to the adiabatic reaction zone for optimal conversion of ethanol and acetaldehyde to 1,3-butadiene.

Thus, according to a preferred embodiment of the process according to the invention, the temperature of the feed before contacting the supported catalyst is in the range of from 320 to 430° C., more preferably from 350 to 410° C., most preferably from 380 to 390° C.

In the process according to the invention, the adiabatic reaction zone comprising a supported catalyst and producing 1,3-butadiene is preferably operated at a pressure of from 0 to 10 barg, more preferably from 1 to 5 barg, most preferably from 1 to 3 barg.

Preferably, too high a temperature drop along the adiabatic reaction zone is avoided.

In a preferred embodiment of the process according to the invention, the process is thus carried out in n adiabatic reaction zones comprising a supported catalyst and producing 1,3-butadiene, wherein n is an integer and is 2 or more, and at least part of the effluent from each (n−1)^{th} adiabatic reaction zone comprising a supported catalyst and producing 1,3-butadiene is fed to the n^{th} adiabatic reaction zone comprising a supported catalyst and producing 1,3-butadiene.

Preferably, an additional feed comprising acetaldehyde is fed to any of the n adiabatic reaction zones comprising a supported catalyst and producing 1,3-butadiene.

More preferably, an additional feed comprising acetaldehyde is fed to each of the n adiabatic reactions zone comprising a supported catalyst and producing 1,3-butadiene.

The n adiabatic reaction zones comprising a supported catalyst and producing 1,3-butadiene are preferably connected in series.

Preferably, the entire effluent from the (n−1)^{th} adiabatic reaction zone comprising a supported catalyst and producing 1,3-butadiene is fed to the n^{th} adiabatic reaction zone comprising a supported catalyst and producing 1,3-butadiene.

More preferably, the additional feed, if present, comprises acetaldehyde and ethanol.

Acetaldehyde may be obtained from the workup of the effluent from a reaction zone or a reactor producing 1,3-butadiene.

The additional feeds that are fed to any of the n adiabatic reaction zones comprising a supported catalyst and producing 1,3-butadiene or are fed to each of the n adiabatic reactions zone comprising a supported catalyst and producing 1,3-butadiene may have the same composition or may have different compositions. Specifically, they may have the same molar ratio of ethanol to acetaldehyde, or a different ratio.

According to a preferred embodiment, additional feed is introduced into each of the n adiabatic reaction zones comprising a supported catalyst and producing 1,3-butadiene.

In all embodiments of the present invention, it is preferred that there are at least two reaction zones, i.e. that n is at least 2.

Direct injection of additional feed into a subsequent reaction zone is disadvantageous, due to the lack of good mixing of feeds (the effluent from the preceding reaction zone with the additional feed), potentially resulting in side reactions just below the feeding point of the additional feed. Therefore, when several reaction zones are separated by a layer of heated inert packing, additional feed is preferable added at the top of the heated inert packing, mixes then in the heated inert packing with the effluent from the preceding reaction zone, and then enters the subsequent reaction zone.

Alternatively, the effluent from a preceding reaction zone and the additional feed may be mixed outside the reactor, i.e. in a pipe, and then the mixture may go to a heat exchanger, or may e.g. go first through a static mixer and then to a heat exchanger.

Preferably, an additional feed comprising acetaldehyde is mixed with (at least parts of) the effluent from the (n−1)^{th} adiabatic reaction zone comprising a supported catalyst and producing 1,3-butadiene, and the mixture is then fed to the n^{th} adiabatic reaction zone comprising a supported catalyst and producing 1,3-butadiene.

Feeds (i.e. the feed comprising ethanol and acetaldehyde, or the mixture with the additional feed comprising acetaldehyde) to the adiabatic reaction zones comprising a supported catalyst and producing 1,3-butadiene preferably are heated to a suitable temperature by heating means before entering the respective adiabatic reaction zone comprising the supported catalyst and producing 1,3-butadiene.

Preferably, the temperature of the feed is higher than 165° C., preferably higher than 200° C., more preferably higher than 250° C., before contacting the supported catalyst.

According to a preferred embodiment of the present invention, the temperature of the feed is in the range of from 320 to 430° C., more preferably from 350 to 410° C., most preferably from 380 to 390° C., before contacting the supported catalyst.

Ideally and preferably, the n adiabatic reaction zones comprising a supported catalyst and producing 1,3-butadiene are connected in series and are operated at the same pressure (as defined above). In practice, however, a slight pressure drop is often observed along the series of n adiabatic reaction zones due to the occurring flow resistance.

Preferably, the effluent from the adiabatic reaction zone (or the last of the n adiabatic reaction zones) comprising a supported catalyst and producing 1,3-butadiene (effluent n) is worked up, to obtain the product 1,3-butadiene.

Kampmeyer et al. (Industrial and Engineering Chemistry, 1949, 41, 3, 550) discloses the use of side streams or auxiliary feeds (multiple point addition and spot addition) in an isothermal process for the production of 1,3-butadiene from ethanol and acetaldehyde. The reaction chamber comprised an insulated electrically-heated stainless steel block. Catalyst temperature was controlled so as to have a variation of only a few degrees along the entire length of the catalyst section of the furnace block and was set to 350° C. The side streams or auxiliary feeds entered first a stream preheater and then an electrically-heated manifold maintained at only 165° C. This temperature would be too low to support an efficient conversion of ethanol and acetaldehyde to 1,3-butadiene by itself.

In the studies underlying the present invention, it was surprisingly found that the use of one or more additional feed(s) in the process according to the invention is particularly advantageous, because the additional feed(s) can be used to deliver heat energy to any of the adiabatic reaction zones comprising a supported catalyst and producing 1,3-butadiene.

The use of one or more additional feed(s) in the process according to the invention is further advantageous, because it allows the recycling of acetaldehyde (and optionally ethanol) fractions separated from the effluents from the adiabatic reaction zones into any of the adiabatic reaction zones comprising a supported catalyst and producing 1,3-butadiene via the additional feed(s), if desired.

Moreover, the presence of n adiabatic reaction zones comprising a supported catalyst and producing 1,3-butadiene, wherein n is an integer and is 2 or more, and of one or more additional feed(s), is further advantageous because it allows a precise adjustment of the composition of the feeds to the adiabatic reaction zones comprising a supported catalyst and producing 1,3-butadiene as required. It is therefore not necessary for the feed to the first adiabatic reaction zone comprising a supported catalyst and producing 1,3-butadiene to comprise a particularly large amount of acetaldehyde, for example, because more acetaldehyde (and optionally ethanol) may be added via the additional feeds after the first adiabatic reaction zone comprising a supported catalyst and producing 1,3-butadiene. As a consequence, a local excess of acetaldehyde undergoing condensation to coke precursors is avoided in the 1,3-butadiene producing reactor. Thus, a decrease of the selectivity to highly undesirable heavy by-products and a more uniform and much slower deactivation of the supported catalysts in the n adiabatic reaction zones comprising a supported catalyst and producing 1,3-butadiene are achieved. This leads to a greater stability, i.e. longer time on stream (TOS), for the supported catalysts, to milder regeneration conditions, as well as to the avoidance of hot spots during the catalyst regeneration procedure.

The addition of additional feeds in the process according to the invention thus maintains catalyst activity, i.e. extends time on stream, so that regeneration of the adiabatic reaction zones only needs to be carried out for a time period in a range of from ⅙ to ½ of the time period for which the catalytic reaction is carried out. This is in contrast to the teaching of WO 2020/126920 A1 and WO 2020/126921 A1, requiring that regeneration must be carried out for a time period of ½ of the duration of the catalytic reaction.

Preferably, regeneration comprises the following subsequent steps:
i. a stripping step, carried out at a temperature in a range of from 300 to 400° C., by contacting the supported catalyst with a gas flow comprising inert gas, the gas flow having an oxygen content of 200 vol.-ppm or less;
ii. a first combustion step carried out at a temperature in a range of from 350 to 400° C., by contacting the supported catalyst with a gas flow comprising inert gas, the gas flow having an oxygen content in a range of from 0.2 to 8 vol. %;
iii. a second combustion step carried out at a temperature in a range of from 400 to 550° C., by contacting the supported catalyst with a gas flow comprising inert gas, the gas flow having an oxygen content in a range of from 0.2 to 8 vol. %;
iv. a stripping step carried out at a temperature in a range of from 550° C. to 300° C., by contacting the supported catalyst with a gas flow comprising inert gas, the gas flow having an oxygen content of less than 200 vol.-ppm;
wherein the gas flows to each of regeneration steps b)i. to b)iv. are first heated and then contact the supported catalyst.

In all embodiments of the invention, the gas used for incorporating oxygen into the gas flow of those regeneration steps that include the feeding of oxygen (namely first combustion step ii., second combustion step iii., or both first combustion step ii. and second combustion step iii.) is conveniently chosen to be air. Air has the advantage that it comprises both an inert gas and oxygen, and that the oxygen can conveniently be dosed to the gas flow, as required in order to supply the desired amount of oxygen to the gas flows comprising oxygen, namely those in regeneration steps i. and ii.

Further details regarding regeneration of the supported catalyst in the adiabatic reaction zone are set out in the application entitled “Adiabatically conducted process for the production of 1,3-butadiene from mixtures of ethanol and acetaldehyde with catalyst regeneration” (PCT application no. PCT/EP2022/058716, attorney reference SH 1657-02WO, filed on even date herewith), the disclosure of which application is incorporated herein in its entirety. Said application entitled “Adiabatically conducted process for the production of 1,3-butadiene from mixtures of ethanol and acetaldehyde with catalyst regeneration” claims priority from European patent application EP21461530.4 filed 1 Apr. 2021, which is also the filing date of European patent application EP21461531.2 (from which the present application claims priority).

The process according to the invention is preferably carried out in two or more adiabatic reaction zones comprising a supported catalyst and producing 1,3-butadiene.

In a preferred embodiment of the process according to the invention, the composition and flow rate of the additional feed are adjusted so as to obtain a molar ratio of ethanol to acetaldehyde in the feed to the n^{th} adiabatic reaction zone comprising a supported catalyst and producing 1,3-butadiene that is equal to 85-115% of the molar ratio of ethanol to acetaldehyde in the feed to the (n−1)^{th} adiabatic reaction zone comprising a supported catalyst and producing 1,3-butadiene.

Preferably, the WHSV in an adiabatic reaction zone comprising a supported catalyst and producing 1,3-butadiene is adjusted such that the molar ratio of ethanol to acetaldehyde in the effluent from this adiabatic reaction zone comprising a supported catalyst and producing 1,3-butadiene is at least 20% higher than the molar ratio of ethanol to acetaldehyde in the feed to this adiabatic reaction zone, more preferably the WHSV in each adiabatic reaction zone comprising a supported catalyst and producing 1,3-butadiene is adjusted such that the molar ratio of ethanol to acetaldehyde in the effluent from this adiabatic reaction zone comprising a supported catalyst and producing 1,3-butadiene is at least 30% higher than the molar ratio of ethanol to acetaldehyde in the feed to this adiabatic reaction zone.

According to a preferred embodiment, the 1,3-butadiene producing reactor includes a first adiabatic reaction zone comprising a supported catalyst and producing 1,3-butadiene and a second adiabatic reaction zone comprising a supported catalyst and producing 1,3-butadiene.

According to another preferred embodiment, the first adiabatic reaction zone comprising a supported catalyst and producing 1,3-butadiene and the second adiabatic reaction zone comprising a supported catalyst and producing 1,3-butadiene are separated by a non-reaction zone, preferably wherein the non-reaction zone is heated, more preferably wherein the heated non-reaction zone comprises an inert packing.

Preferably, the inert packing is selected from the group consisting of silicon carbide, inert ceramic beds, ceramic beads, extrudates, rings with a diameter of 2-7 mm, stainless steel mesh, foams, and mixtures thereof.

Preferably, at least part of the effluent from the first adiabatic reaction zone comprising a supported catalyst and producing 1,3-butadiene is passed through the non-reaction zone and is then fed into the second adiabatic reaction zone comprising a supported catalyst and producing 1,3-butadiene.

As outlined above, the progress of the endothermic conversion of the mixture of ethanol and acetaldehyde to 1,3-butadiene causes a temperature drop along the adiabatic reaction zones comprising a supported catalyst and producing 1,3-butadiene. The effluent from the first adiabatic reaction zone comprising a supported catalyst and producing 1,3-butadiene thus has a lower temperature than the feed comprising ethanol and acetaldehyde to the first adiabatic reaction zone comprising a supported catalyst and producing 1,3-butadiene. Hence, it is advantageous that the non-reaction zone separating the first and the second adiabatic reaction zones comprising a supported catalyst and producing 1,3-butadiene is heated, to ensure that the feed to the second adiabatic reaction zone comprising a supported catalyst and producing 1,3-butadiene has a sufficiently high temperature to deliver the energy required for the conversion of ethanol and acetaldehyde to 1,3-butadiene in the second adiabatic reaction zone comprising a supported catalyst and producing 1,3-butadiene.

According to a preferred embodiment, the temperature of the feed before contacting the supported catalyst of the first adiabatic reaction zone producing 1,3-butadiene is in the range of from 320 to 430° C., more preferably from 350 to 410° C., most preferably from 380 to 390° C.

According to another preferred embodiment, the temperature of the feed before contacting the supported catalyst of the second adiabatic reaction zone producing 1,3-butadiene is in the range of from 320 to 430° C., more preferably from 350 to 410° C., most preferably from 380 to 390° C.

Preferably, the first adiabatic reaction zone comprising a supported catalyst and producing 1,3-butadiene is operated at a pressure of from 0 to 10 barg, more preferably from 1 to 5 barg, most preferably from 1 to 3 barg.

Preferably, the second adiabatic reaction zone comprising a supported catalyst and producing 1,3-butadiene is operated at a pressure of from 0 to 10 barg, more preferably from 1 to 5 barg, most preferably from 1 to 3 barg.

Most preferably, the first and the second adiabatic reaction zones comprising a supported catalyst and producing 1,3-butadiene are operated at the same pressure (as defined above).

According to another preferred embodiment, the first adiabatic reaction zone comprising a supported catalyst and producing 1,3-butadiene and the second adiabatic reaction zone comprising a supported catalyst and producing 1,3-butadiene are separated by a non-reaction zone, and at least part of the effluent from the first adiabatic reaction zone comprising a supported catalyst and producing 1,3-butadiene is passed through a heat exchanger and is then fed into the second adiabatic reaction zone comprising a supported catalyst and producing 1,3-butadiene.

Preferably, the non-reaction zone comprises an inert packing.

Most preferably, the inert packing is selected from the group consisting of silicon carbide, inert ceramic beds, ceramic beads, extrudates, rings with a diameter of 2-7 mm, stainless steel mesh, foams, and mixtures thereof.

The heat exchanger between the first adiabatic reaction zone and the second adiabatic reaction zone fulfils the same function as described above for the heated non-reaction zone.

Preferably, an additional feed comprising acetaldehyde is fed into the reactor after the first adiabatic reaction zone comprising a supported catalyst and producing 1,3-butadiene, more preferably the additional feed is mixed with the effluent from the first adiabatic reaction zone comprising a supported catalyst and producing 1,3-butadiene and is then fed to the second adiabatic reaction zone comprising a supported catalyst and producing 1,3-butadiene.

According to a preferred embodiment, the additional feed comprises acetaldehyde and ethanol.

In a preferred embodiment of the process according to the invention, the additional feed further comprises ethanol, and the molar ratio of ethanol to acetaldehyde in the additional feed is in the range of from 0.1 to 5, preferably 1 to 2, more preferably 1.4 to 1.8.

According to a preferred embodiment of the process according to the invention, a first 1,3-butadiene producing reactor having at least a first adiabatic reaction zone comprising a supported catalyst and producing 1,3-butadiene, and a second 1,3-butadiene producing reactor having at least a second adiabatic reaction zone comprising a supported catalyst and producing 1,3-butadiene are connected in series, and at least part of the effluent from the first 1,3-butadiene producing reactor is fed to the second 1,3-butadiene producing reactor, more preferably an additional feed comprising acetaldehyde is fed into the second reactor.

Preferably, the entire effluent from the first 1,3-butadiene producing reactor is fed to the second 1,3-butadiene producing reactor.

More preferably, an additional feed comprising acetaldehyde and ethanol is fed into the second reactor.

According to a preferred embodiment of the process according to the invention, the effluent from the first 1,3-butadiene producing reactor is heated and is then fed to the second 1,3-butadiene producing reactor.

Thus, according to a preferred embodiment of the present invention, the temperature of the feed to the second 1,3-butadiene producing reactor, comprising at least parts of the effluent from the first 1,3-butadiene producing reactor and optionally an additional feed, is in the range of from 320 to 430° C., more preferably from 350 to 410° C., most preferably from 380 to 390° C. before entering the second 1,3-butadiene producing reactor.

According to a second aspect of the invention, the process for the production of 1,3-butadiene from ethanol comprises
i. producing acetaldehyde from ethanol in an acetaldehyde producing reactor having a reaction zone, the reaction zone of the acetaldehyde producing reactor comprising a supported or unsupported (bulk) catalyst, and
ii. producing 1,3-butadiene according to the process as defined herein.

Preferably, the reaction zone of the acetaldehyde producing reactor is an isothermal reaction zone.

Said process for the production of 1,3-butadiene from ethanol is particularly advantageous, because the acetaldehyde required in step ii. can be generated from ethanol and does not have to be purchased as a raw material for the process according to the invention.

According to a preferred embodiment of the process according to the invention, the supported or unsupported (bulk) catalyst comprises one or more of zinc, copper, silver, chromium, magnesium and nickel, in particular one or more of zinc and copper.

Preferably, the acetaldehyde producing reactor comprises a supported catalyst.

According to a preferred embodiment, the support of the supported catalyst of the acetaldehyde producing reactor is selected from the group consisting of ordered and non-ordered porous silica supports, aluminium oxide supports, aluminosilicate supports, clays, other porous oxide supports, and mixtures thereof.

Preferably, the support of the supported catalyst of the acetaldehyde producing reactor is a silica support, more preferably an ordered or non-ordered porous silica support.

Preferably, the support of the supported catalyst of the acetaldehyde producing reactor has a specific surface area (SSA) in a range of from 7 to 550 m²/g, more preferably in a range of from 190 to 350 m²/g.

Preferably, the support of the supported catalyst of the acetaldehyde producing reactor has an average pore diameter in a range of from 10 to 300 Å (determined by the method of Barrett, Joyner and Halenda).

Preferably, the support of the supported catalyst of the acetaldehyde producing reactor has a pore volume in a range of from 0.2 to 1.5 ml/g (determined by the method of Barrett, Joyner and Halenda).

More preferably, the support of the supported catalyst of the acetaldehyde producing reactor is a silica support with a specific surface area in a range of from 7 to 550 m²/g, most preferably from 190 to 350 m²/g, and an average pore diameter in a range of from 10 to 300 Å, and a pore volume in a range of from 0.2 to 1.5 ml/g.

Most preferably, the support of the supported catalyst of the acetaldehyde producing reactor is an ordered or non-ordered porous silica support with a specific surface area in a range of from 7 to 550 m²/g, most preferably from 190 to 350 m²/g, and an average pore diameter in a range of from 10 to 300 Å, and a pore volume in a range of from 0.2 to 1.5 ml/g.

The supported or unsupported (bulk) catalyst in the reaction zone of the acetaldehyde producing reactor may be any (commercial) catalyst that is able to catalyse the dehydrogenation of ethanol to acetaldehyde.

### 2) Plant for the Production of 1,3-Butadiene

A third aspect of the present invention relates to a plant for the production of 1,3-butadiene comprising at least one reactor for producing 1,3-butadiene, the reactor for producing 1,3-butadiene having
a) at least one zone for producing 1,3-butadiene, the zone comprising a supported catalyst for producing 1,3-butadiene from ethanol and acetaldehyde, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react the ethanol and the acetaldehyde under adiabatic conditions,
the reactor for producing 1,3-butadiene further having
c) means for regenerating the supported catalyst for producing 1,3-butadiene, preferably wherein the means for regenerating the supported catalyst for producing 1,3-butadiene comprise
x) means for feeding a flow comprising inert gas into the reactor for producing 1,3-butadiene, and
y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.

A fourth aspect of the present invention relates to a plant for the production of 1,3-butadiene from ethanol, comprising
i. at least one reactor for producing acetaldehyde from ethanol, the reactor for producing acetaldehyde from ethanol having
   a) at least one zone for producing acetaldehyde from ethanol, the zone for producing acetaldehyde from ethanol comprising a supported or unsupported (bulk) catalyst for producing acetaldehyde, and
   b) means for feeding a feed comprising ethanol into the reactor for producing acetaldehyde; and
ii. at least one reactor for producing 1,3-butadiene, the reactor for producing 1,3-butadiene having
   a) at least one zone for producing 1,3-butadiene, the zone comprising a supported catalyst for producing 1,3-butadiene from ethanol and acetaldehyde, and
   b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react ethanol and acetaldehyde under adiabatic conditions,
the reactor for producing 1,3-butadiene further having
c) means for regenerating the supported catalyst for producing 1,3-butadiene, preferably wherein the means for regenerating the supported catalyst for producing 1,3-butadiene comprise
   x) means for feeding a flow comprising inert gas into the reactor for producing 1,3-butadiene, and
   y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.

Preferably, the reaction zone of the acetaldehyde producing reactor is an isothermal reaction zone.

Preferred embodiments of the processes for the production of 1,3-butadiene according to the invention correspond to or can be derived from preferred embodiments of the plants according to the invention, and vice versa.

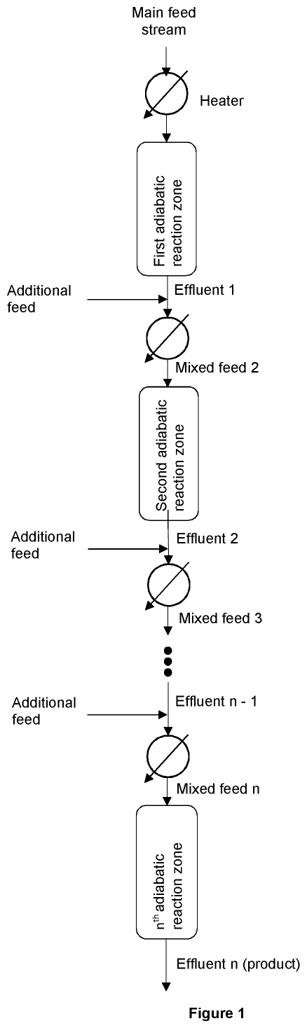

FIG. 1: Scheme of an exemplary process for the production of 1,3-butadiene according to the invention

The following examples show the advantages of the present invention. Unless noted otherwise, all percentages are given by weight.

### EXAMPLES

All tests were carried out in a 52×3000 mm tube reactor (inner diameter×length) loaded with a supported tantalum catalyst (3 wt. % Ta₂O₅/SiO₂, with wt. % of tantalum oxide calculated as Ta₂O₅ based on the total weight of the catalyst). Examples 1 to 5 were carried out in the reactor loaded with 2.4 kg of the catalyst (length of the catalytic bed 2400 mm, bed volume 5.1 dm³). Example 6 was carried out in the reactor loaded with two catalytic beds of 900 mm length, separated by 600 mm of carborundum as an inert packing (total weight of catalytic beds 1.8 kg, total beds volume 3.8 dm³).

The effluent from the reactor was analyzed using an online GC/MS system. The experimental conditions and results are shown in Table 1 below.

WHSV, conversion, selectivity and yield were calculated as follows:

WHSV (one catalytic bed, no additional feeds)=mass flow rate of feed/mass of catalyst

WHSV (first catalytic bed)=mass flow rate of main feed/mass of catalyst in first catalytic bed
WHSV (second catalytic bed)=(mass flow rate of main feed+mass flow rate of additional feed)/mass of catalyst in second catalytic bed

Conversion=(moles of converted reactants/moles of feed)−100

Selectivity=(C moles in 1,3-butadiene/C moles in all products)−100

Yield=(conversion−selectivity)/100

### Example 1

A feed stream comprising aqueous ethanol (94 wt. %) and acetaldehyde in a molar ratio of ethanol:acetaldehyde=2.2 was heated and fed to the reactor with a WHSV of 2.0 h⁻¹. The temperature at the inlet to the catalytic bed was 410° C. The reactor was operated at 1.8 barg. Heat was supplied to the catalytic bed only by the feed, hence the temperature at the reactor outlet was 300° C.

### Example 2

The reaction was carried out as in Example 1, except that the temperature at the inlet to the catalytic bed was 390° C.

### Example 3

The reaction was carried out as in Example 1, except that the temperature at the inlet to the catalytic bed was 380° C.

### Example 4

A feed stream comprising aqueous ethanol (94 wt. %) and acetaldehyde in a molar ratio of ethanol acetaldehyde=3.6 was heated and fed to the reactor with a WHSV of 2.0 h⁻¹. The temperature at the inlet to the catalytic bed was 380° C. The reactor was operated at 1.8 barg.

### Example 5

The reaction was carried out as in Example 4, except that the molar ratio ethanol:acetaldehyde in the feed was 2.9.

### Example 6

A main feed comprising aqueous ethanol (94 wt. %) and acetaldehyde in a molar ratio of ethanol acetaldehyde=2.9 was heated and fed to the reactor with a WHSV of 3.0 h⁻¹. The temperature at the inlet to the first catalytic bed was 380° C. The reactor was operated at 1.8 barg. Pre-heated additional feed comprising aqueous ethanol (94 wt. %) and acetaldehyde in a molar ratio of ethanol:acetaldehyde=1.6 was added to the reactor at the top of the inert packing between the two catalytic beds. The mixed feed (effluent from the first catalytic bed+additional feed) was heated along the inert packing to reach the temperature of 380° C. at the inlet to the second catalytic bed. The WHSV of the second catalytic bed was 4.1 h⁻¹. Heat was supplied to the catalytic beds only by the respective feeds.

TABLE 1

| | Main feed | Additional | | | | | | |
|---|---|---|---|---|---|---|---|---|
| | EtOH/ | feed | | | | | | |
| | AcH | EtOH/AcH | | | | | Selectivity | Yield of |
| | ratio | ratio | Tᵢₙₗₑₜ | WHSV | TOS | Conversion | to 1,3- | 1,3-BDN |
| Ex. | [mol/mol] | [mol/mol] | [° C.] | [h⁻¹]ª | [h] | [%]^{b} | BDN [%]^{b} | [%]^{b} |
| 1 | 2.2 | n/a | 410 | 2.0 | 20 | 38 | 70 | 26.6 |
| 2 | 2.2 | n/a | 390 | 2.0 | 20 | 42 | 72 | 30.2 |
| 3 | 2.2 | n/a | 380 | 2.0 | 20 | 42 | 74 | 31.1 |
| | | | | | 100 | 36 | 71 | 25.6 |
| 4 | 3.6 | n/a | 380 | 2.0 | 20 | 32 | 69 | 22.1 |
| 5 | 2.9 | n/a | 380 | 2.0 | 20 | 38 | 72 | 27.4 |
| | | | | | 100 | 36 | 71 | 25.6 |
| 6 | 2.9 | 1.6 | 380 | 3.0/4.1 | 20 | 44 | 73 | 32.1 |
| | | | | | 100 | 42 | 72 | 30.2 |

^{a}for the first catalytic bed or for the first catalytic bed/second catalytic bed
^{b}in average for a given time on stream
EtOH = ethanol
AcH = acetaldehyde
1,3-BDN = 1,3-butadiene
T = temperature
WHSV = weight hourly space velocity
TOS = time on stream

1. A process for the production of 1,3-butadiene comprising reacting a feed comprising ethanol and acetaldehyde in a 1,3-butadiene producing reactor having at least one adiabatic reaction zone, the adiabatic reaction zone comprising a supported catalyst and producing 1,3-butadiene.

2. The process according to claim 1, wherein the feed comprises at least 40 wt. % of ethanol based on the total weight of the feed.

3. The process according to claim 1 or claim 2, wherein the feed comprises at least 12.5 wt. % of acetaldehyde based on the total weight of the feed.

4. The process according to any of the preceding claims, wherein the supported catalyst comprises one or more of tantalum, zirconium, niobium, hafnium, titanium, and tin, in particular tantalum,
preferably wherein the supported catalyst comprises tantalum in an amount of from 0.1 to 10 wt. %, preferably from 0.5 to 5 wt. %, more preferably from 2 to 3 wt. %, calculated as Ta₂O₅ and based on the total weight of the supported catalyst.

5. The process according to any of the preceding claims, wherein the molar ratio of ethanol to acetaldehyde in the feed is in the range of from 1 to 7, preferably 1.5 to 5, more preferably 2 to 4, in particular 2.5 to 3.5, such as about 3.

6. The process according to any of the preceding claims, wherein the temperature of the feed before contacting the supported catalyst is in the range of from 320 to 430° C.

7. The process according to any of the preceding claims, wherein the adiabatic reaction zone comprising a supported catalyst and producing 1,3-butadiene is operated at a pressure of from 0 to 10 barg,
preferably from 1 to 5 barg, more preferably from 1 to 3 barg.

8. The process according to any of the preceding claims, wherein the process is carried out in n adiabatic reaction zones comprising a supported catalyst and producing 1,3-butadiene, wherein n is an integer and is 2 or more, and at least part of the effluent from each (n−1)^{th} adiabatic reaction zone comprising a supported catalyst and producing 1,3-butadiene is fed to the n^{th} adiabatic reaction zone comprising a supported catalyst and producing 1,3-butadiene,
preferably wherein an additional feed comprising acetaldehyde is fed to any of the n adiabatic reaction zones comprising a supported catalyst and producing 1,3-butadiene,
more preferably wherein an additional feed comprising acetaldehyde is fed to each of the n adiabatic reaction zone comprising a supported catalyst and producing 1,3-butadiene.

9. The process according to claim 8, wherein composition and flow rate of the additional feed are adjusted so as to obtain a molar ratio of ethanol to acetaldehyde in the feed to the n^{th} adiabatic reaction zone comprising a supported catalyst and producing 1,3-butadiene that is equal to 85-115% of the molar ratio of ethanol to acetaldehyde in the feed to the (n−1)^{th} adiabatic reaction zone comprising a supported catalyst and producing 1,3-butadiene.

10. The process according to claim 8 or 9, wherein the WHSV in an adiabatic reaction zone comprising a supported catalyst and producing 1,3-butadiene is adjusted such that the molar ratio of ethanol to acetaldehyde in the effluent from this adiabatic reaction zone comprising a supported catalyst and producing 1,3-butadiene is at least 20% higher than the molar ratio of ethanol to acetaldehyde in the feed to this adiabatic reaction zone,
preferably wherein the WHSV in each adiabatic reaction zone comprising a supported catalyst and producing 1,3-butadiene is adjusted such that the molar ratio of ethanol to acetaldehyde in the effluent from this adiabatic reaction zone comprising a supported catalyst and producing 1,3-butadiene is at least 30% higher than the molar ratio of ethanol to acetaldehyde in the feed to this adiabatic reaction zone.

11. The process according to any of the preceding claims, wherein the 1,3-butadiene producing reactor includes a first adiabatic reaction zone comprising a supported catalyst and producing 1,3-butadiene and a second adiabatic reaction zone comprising a supported catalyst and producing 1,3-butadiene.

12. The process according to claim 11, wherein the first adiabatic reaction zone comprising a supported catalyst and producing 1,3-butadiene and the second adiabatic reaction zone comprising a supported catalyst and producing 1,3-butadiene are separated by a non-reaction zone,
preferably wherein the non-reaction zone is heated,
more preferably wherein the heated non-reaction zone comprises an inert packing.

13. The process according to claim 11, wherein the first adiabatic reaction zone comprising a supported catalyst and producing 1,3-butadiene and the second adiabatic reaction zone comprising a supported catalyst and producing 1,3-butadiene are separated by a non-reaction zone, and wherein at least part of the effluent from the first adiabatic reaction zone comprising a supported catalyst and producing 1,3-butadiene is passed through a heat exchanger and is then fed into the second adiabatic reaction zone comprising a supported catalyst and producing 1,3-butadiene.

14. The process according to any of the claims 11 to 13, wherein an additional feed comprising acetaldehyde is fed into the reactor after the first adiabatic reaction zone comprising a supported catalyst and producing 1,3-butadiene,
preferably wherein the additional feed is mixed with the effluent from the first adiabatic reaction zone comprising a supported catalyst and producing 1,3-butadiene and is then fed to the second adiabatic reaction zone comprising a supported catalyst and producing 1,3-butadiene.

15. The process of claim 14, wherein the additional feed further comprises ethanol, and the molar ratio of ethanol to acetaldehyde in the additional feed is in the range of from 0.1 to 5, preferably 1 to 2, more preferably 1.4 to 1.8.

16. The process according to any of the preceding claims, wherein a first 1,3-butadiene producing reactor having at least a first adiabatic reaction zone comprising a supported catalyst and producing 1,3-butadiene, and a second 1,3-butadiene producing reactor having at least a second adiabatic reaction zone comprising a supported catalyst and producing 1,3-butadiene are connected in series, and at least part of the effluent from the first 1,3-butadiene producing reactor is fed to the second 1,3-butadiene producing reactor,
more preferably wherein an additional feed comprising acetaldehyde is fed into the second reactor.

17. The process according to claim 16, wherein the effluent from the first 1,3-butadiene producing reactor is heated and is then fed to the second 1,3-butadiene producing reactor.

18. A process for the production of 1,3-butadiene from ethanol, comprising
i. producing acetaldehyde from ethanol in an acetaldehyde producing reactor having a reaction zone, the reaction zone of the acetaldehyde producing reactor comprising a supported or unsupported (bulk) catalyst, and
ii. producing 1,3-butadiene according to the process of any of the preceding claims,
preferably wherein the reaction zone of the acetaldehyde producing reactor is an isothermal reaction zone.

19. A plant for the production of 1,3-butadiene comprising at least one reactor for producing 1,3-butadiene, the reactor for producing 1,3-butadiene having
a) at least one zone for producing 1,3-butadiene, the zone comprising a supported catalyst for producing 1,3-butadiene from ethanol and acetaldehyde, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react the ethanol and the acetaldehyde under adiabatic conditions,
the reactor for producing 1,3-butadiene further having
c) means for regenerating the supported catalyst for producing 1,3-butadiene, preferably wherein the means for regenerating the supported catalyst for producing 1,3-butadiene comprise
x) means for feeding a flow comprising inert gas into the reactor for producing 1,3-butadiene, and
y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions.

20. A plant for the production of 1,3-butadiene from ethanol, comprising
i. at least one reactor for producing acetaldehyde from ethanol, the reactor for producing acetaldehyde from ethanol having
a) at least one zone for producing acetaldehyde from ethanol, the zone for producing acetaldehyde from ethanol comprising a supported or unsupported (bulk) catalyst for producing acetaldehyde, and
b) means for feeding a feed comprising ethanol into the reactor for producing acetaldehyde; and
ii. at least one reactor for producing 1,3-butadiene, the reactor for producing 1,3-butadiene having
a) at least one zone for producing 1,3-butadiene, the zone comprising a supported catalyst for producing 1,3-butadiene from ethanol and acetaldehyde, and
b) means for feeding a feed comprising ethanol and acetaldehyde into the reactor for producing 1,3-butadiene,
the reactor for producing 1,3-butadiene having reactant heating means for heating the feed comprising ethanol and acetaldehyde before contacting the supported catalyst for producing 1,3-butadiene, the reactant heating means being sufficient to react ethanol and acetaldehyde under adiabatic conditions,
the reactor for producing 1,3-butadiene further having
c) means for regenerating the supported catalyst for producing 1,3-butadiene,
preferably wherein the means for regenerating the supported catalyst for producing 1,3-butadiene comprise
x) means for feeding a flow comprising inert gas into the reactor for producing 1,3-butadiene, and
y) means for feeding a flow comprising oxygen into the reactor for producing 1,3-butadiene,
the reactor for producing 1,3-butadiene having regenerant heating means for heating a flow comprising the inert gas and the oxygen before contacting the supported catalyst for producing 1,3-butadiene, the regenerant heating means being sufficient to regenerate the supported catalyst under adiabatic conditions,
preferably wherein the reaction zone of the acetaldehyde producing reactor is an isothermal reaction zone.

21. The plant according to claim 19 or 20, wherein the supported catalyst for producing 1,3-butadiene from ethanol and acetaldehyde comprises one or more of tantalum, niobium, hafnium, and tin,
preferably wherein the supported catalyst for producing 1,3-butadiene from ethanol and acetaldehyde comprises tantalum.
